(19) United States
(12) Patent Application Publication
(10) Pub. No.: US 2007/0243573 A1
(43) Pub. Date: Oct. 18, 2007

(54) METHOD AND APPARATUS FOR IMMOBILIZING CELLS, AND CELL-IMMOBILIZED SUBSTRATE

(76) Inventors: Kimio SUMARU, Tsukuba-shi (JP); Junichi EDAHIRO, Abiko-shi (JP); Yuki OOSHIMA, Amagasaki-shi (JP); Yuuichi TADA, Fujinomiya-shi (JP); Shinji SUGIURA, Tsukuba-shi (JP); Toshiyuki KANAMORI, Tsukuba-shi (JP)

Correspondence Address: OSTROLENK FABER GERB & SOFFEN, 1180 AVENUE OF THE AMERICAS, NEW YORK, NY, 100368403, US

(21) Appl. No.: 11/673,361

(22) Filed: Feb. 9, 2007

(30) Foreign Application Priority Data
Feb. 14, 2006 (JP) 2006-036646

(51) Int. Cl. C12Q 1/02; C12M 1/42; C12N 11/14; C12N 13/00

(52) U.S. Cl. 435/29; 435/173.4; 435/176; 435/243; 435/283.1

(57) ABSTRACT

A method and apparatus for efficiently immobilizing cells on a substrate without damaging the cells, and a cell-immobilized substrate is provided. Cells 4a contacting a substrate 2 is irradiated with light 10 which includes light having a wavelength of 330 to 410 nm, thereby adhering cells 4a to the substrate 2.

The present application claims priority on Japanese Patent Application No. 2006-36646, filed Feb. 14, 2006, the content of which is incorporated herein by reference.

### TECHNICAL FIELD

The present invention relates to a cell-immobilized substrate for use in confirming the influence of a drug on cells such as animal cells. Further, the present invention also relates to a method and apparatus for immobilizing cells, which is applicable to the manufacture of such a cell-immobilized substrate. Furthermore, the present invention also relates to a test method using the cell-immobilized substrate, and a method for sorting cells.

### BACKGROUND ART

In the study of cells such as animal cells, cells are cultured under specific environmental conditions, and the influence of the environmental conditions on the cells is evaluated. For example, drug screening for confirming the influence of a drug on cells is an essential technique in the development of a new drug.

In this technique, a cell-immobilized substrate obtained by immobilizing cells on a substrate is used (for example, see Patent Document 1).

As techniques for immobilizing cells on a substrate, a method is known in which target cells are adhered to a substrate through antibodies which specifically bind to the target cells, and a method in which target cells are immobilized on a substrate through an organic compound membrane (for example, see Patent Document 2).

[Patent Document 1] Japanese Unexamined Patent Application, First Publication No. 2005-46121

[Patent Document 2] Japanese Unexamined Patent Application, First Publication No. Hei 10-123031

### SUMMARY OF THE INVENTION

However, in the prior art, for immobilizing cells on a substrate, it was necessary to adhere antibodies to a substrate or form an organic compound membrane in advance. Therefore, immobilization of cells was laborious.

On the other hand, when a cell-immobilized substrate is used for drug screening or the like, it is required that the cells are in a physiologically normal state. Therefore, it is necessary that cells not be physiologically damaged during immobilization.

In the techniques using antibodies or an organic compound membrane, it was sometimes difficult to accurately evaluate the influence of a drug on cells because the antibodies or organic compound membrane affected the physiological state of the cells.

Further, in recent years, tailor-made medical treatments, which take into consideration individual differences of drug sensitivity, has been attracting attention. In tailor-made medical treatments, studies have been conducted on the use of cell-immobilized substrates. However, for popularizing tailor-made medical treatments, lowering of the cost is indispensable. Therefore, an efficient method for immobilizing cells has been desired.

The present invention has been achieved taking into consideration of the above circumstances, with various objects including the following:

(i) to provide a method and apparatus for efficiently immobilizing cells on a substrate without damaging the cells, a cell-immobilized substrate, a testing method and a method for sorting cells; and

(ii) to provide a method and apparatus for immobilizing cells on a substrate, cell-immobilized substrate and test method, which enable accurate evaluation in testing the action of a drug on cells.

Specifically, the present invention adopts various embodiments including the following:

(1) A method for immobilizing cells by adhering the cells to a surface of a substrate, including: irradiating cells with light while contacting the cells to a surface of a substrate, thereby adhering the cells to the substrate, the light including light having a wavelength of 330 to 410 nm.

(2) The method according to item (1) above, wherein the light has an irradiation energy of 1 to 100 J/cm².

(3) The method according to item (1) above, wherein the cells are irradiated with the light in the presence of a serum.

(4) The method according to item (1) above, wherein at least the surface of the substrate includes a non-photoresponsive material.

(5) The method according to item (4) above, wherein at least the surface of the substrate includes polystyrene.

(6) A cell-immobilized substrate in which cells have been immobilized by the method of any one of items (1) to (5) above.

(7) An apparatus for immobilizing cells by adhering the cells to a surface of a substrate, the apparatus being provided with an irradiation unit for irradiating a desired region of the substrate, the irradiation unit irradiating light to cells which are in contact with the surface of the substrate, thereby adhering the cells to the substrate, the light including light having a wavelength of 330 to 410 nm.

(8) The apparatus according to item (7) above, wherein the irradiation unit includes a light source and a reflection device, the reflection device reflecting light generated from the light source to irradiate a desired region of the substrate.

(9) A method for testing the action of drug on cells using the cell-immobilized substrate of item (6) above, including: contacting a drug with the cells; and detecting the action of the drug on the cells.

(10) A method for sorting some cells from a plurality of types of cells, including: leading a plurality of types of cells to a surface of a substrate; selectively irradiating target cells with light including light having a wavelength of 330 to 410 nm while contacting the target cells to the surface of the substrate, thereby adhering the target cells to the substrate; and removing cells other than the target cells from the surface of the substrate.

### BRIEF DESCRIPTION OF THE DRAWINGS

FIG. 1 is a block diagram showing an example of a cell-immobilized substrate according to the present invention.

FIG. 2 is a schematic diagram showing a manufacturing method of the cell-immobilized substrate shown in FIG. 1.

FIG. 3 is an explanatory diagram showing adhesion of cells to a substrate in the manufacturing method of cell-immobilized substrate shown in FIG. 1.

FIG. 4 is a schematic diagram following the scheme shown in FIG. 2.

FIG. 5 is a schematic diagram following the scheme shown in FIG. 4.

FIG. 6 is a schematic diagram following the scheme shown in FIG. 5.

FIG. 7 is an example of a cell-immobilizing apparatus applicable to the method for immobilizing cells according to the present invention.

FIG. 8 is an explanatory diagram showing an example of a method for using the cell-immobilized substrate shown in FIG. 1.

FIG. 9 is an explanatory diagram showing a method for detecting a reaction between cells and a drug, using the cell-immobilized substrate shown in FIG. 1.

FIG. 10 is an explanatory diagram showing an example of the method for sorting cells according to the present invention.

FIG. 11 is an explanatory diagram showing another example of the method for sorting cells according to the present invention.

FIG. 12 is an explanatory diagram showing still another example of the method for sorting cells according to the present invention.

FIG. 13 is a block diagram showing an example of an apparatus applicable to the method for sorting cells according to the present invention.

FIG. 14 is a graph showing the test results of the working examples with respect to the influence of irradiation energy of light on the proliferation ability of cells.

FIG. 15 is a photograph of a flow channel used in a working example in which cells have been immobilized at a predetermined position by light irradiation.

FIG. 16 is a photograph of a flow channel used in another working example in which cells of a different type have been immobilized at a predetermined position by a similar procedure following the procedure shown in FIG. 15.

FIG. 17 is a photograph of a surface of a substrate used in still another working example in which cells have been immobilized on the substrate surface by light irradiation.

FIG. 18 is a photograph of a surface of a substrate used in still another working example in which cells have been immobilized on the substrate surface by light irradiation.

FIG. 19 is a photograph of a surface of a substrate used in still another working example in which cells have been immobilized on the substrate surface by light irradiation.

FIG. 20 is a photograph of a surface of a substrate used in still another working example in which cells have been immobilized on the substrate surface by light irradiation.

FIG. 21 is a photograph of a surface of a substrate used in still another working example in which cells have been immobilized on the substrate surface by light irradiation.

FIG. 22 is a photograph of a surface of a substrate used in still another working example in which cells have been immobilized on the substrate surface by light irradiation.

FIG. 23 is a photograph of a surface of a substrate used in still another working example in which cells have been immobilized on the substrate surface by light irradiation.

FIG. 24 is a photograph of a surface of a substrate used in still another working example in which cells have been immobilized on the substrate surface by light irradiation.

FIG. 25 is a photograph of a surface of a substrate used in still another working example in which cells have been immobilized on the substrate surface by light irradiation.

### REFERENCE NUMERALS

1 Cell array (cell-immobilized substrate)
2, 32, 52 Substrate
3a to 3d First through fourth flow channel
4a to 4d, 34, 34a to 34c, 44, 46, 50 Cells
6a to 6d, 7a to 7d, 8a to 8d, 9a to 9d Irradiating portion
11a to 11d First through fourth drug-containing liquid
12 Detection unit
22 Irradiation unit
25 Digital micromirror device (reflection device)

### BEST MODE FOR CARRYING OUT THE INVENTION

Hereinbelow, the present invention will be described in detail.

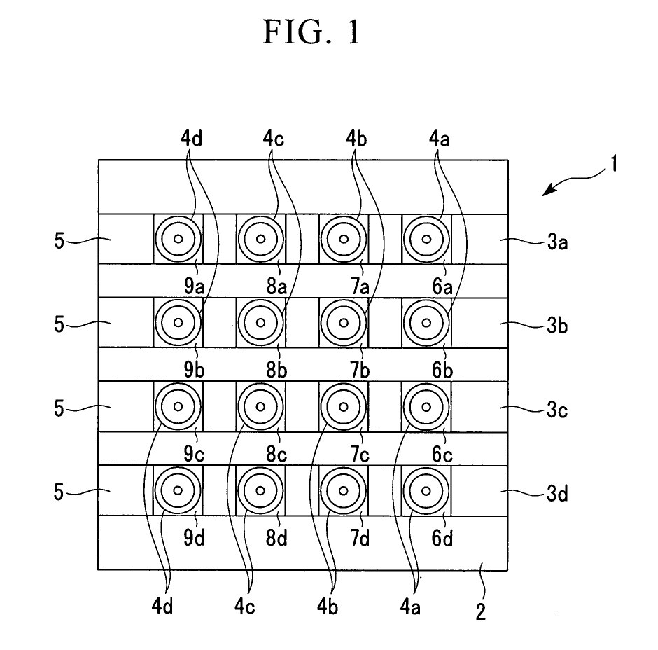

FIG. 1 shows a cell array 1 which is an example of a cell-immobilized substrate according to the present invention.

The cell array 1 shown in FIG. 1 has four flow channels 3a to 3d (first through fourth channels) formed in a substrate 2. In the first through fourth flow channels 3a to 3d, first through fourth cells 4a to 4d are immobilized.

The material for the substrate 2 is not particularly limited, and examples include synthesized resins, glass, metals and silicon.

Preferred examples of synthesized resins include polystyrene resins, silicone resins (such as a polydimethylsiloxane resin), acrylic resins (such as a methyl polymethacrylate resin), polyethylene resins, polypropylene resins, polycarbonate resins and epoxy resins.

The substrate 2 may be made of any material in which at least the surface thereof is made of any of the above-exemplified materials. For example, the substrate 2 may have the surface made of any of the above-exemplified materials and the remainder made of other materials.

The substrate 2 is preferably made of a material capable of transmitting irradiation light (explained below).

A material in which the molecular structure is changed by light is called a “photoresponsive material”. However, as the substrate 2, a material which does not exhibit a photoresponsive property (i.e., a non-photoresponsive material) may be used. Examples of non-photoresponsive materials include the above-exemplified materials (i.e., synthesized resins, glass, metals, silicon, and the like).

The adhesiveness of the substrate 2 can be enhanced by a surface treatment. Preferable examples of surface treatment methods include treatment methods in which polar functional groups (e.g., —OH, —NH₂, —COOH) can be formed on the surface of the substrate 2, such as plasma treatment, ozone treatment, corona treatment, and flame treatment.

Especially, a tissue culture polystyrene (TCPS), which is a plasma-treated or ozone-treated polystyrene, is particularly desirable.

The substrate 2 may be provided with a coating layer composed of a cell-adhesive component. As a cell-adhesive component, one or more of fibronectin, vitronectin and laminin can be used. By forming a coating layer of fibronectin or the like, the adhesion strength of cells to the substrate 2 can be enhanced. The reason why the cell adhesion property can be enhanced by the coating layer is presumed that the superstructure of the membrane protein (such as integrin) on the cell surface is changed by light, so that the cell surface is strongly bonded to the ligand of the coating layer component (e.g., fibronectin).

The cross-sectional shape of the first through fourth flow channels 3a to 3d is not particularly limited, and the shape may be a rectangle, a triangle, a trapezoid, a circle, a semicircle, an ellipse, or the like. In the shown example, the plane view of the flow channels 3a to 3d has a rectilinear shape, and the flow channels 3a to 3d are formed in parallel to each other.

The flow channels 3a to 3d are preferably slits formed on the substrate in which a covering material is arranged. Further, the flow channels 3a to 3d are preferably closed channels.

As shown in FIG. 1, in the inner space of the first flow channel 3a, first through fourth cells 4a to 4d are arranged and immoblizied in the lengthwise direction of the flow channel 3a. Likewise, in the inner spaces of the second through fourth flow channels 3b to 3d, first through fourth cells 4a to 4d are immobilized.

Next, explanation is given on the manufacturing method of a cell array 1.

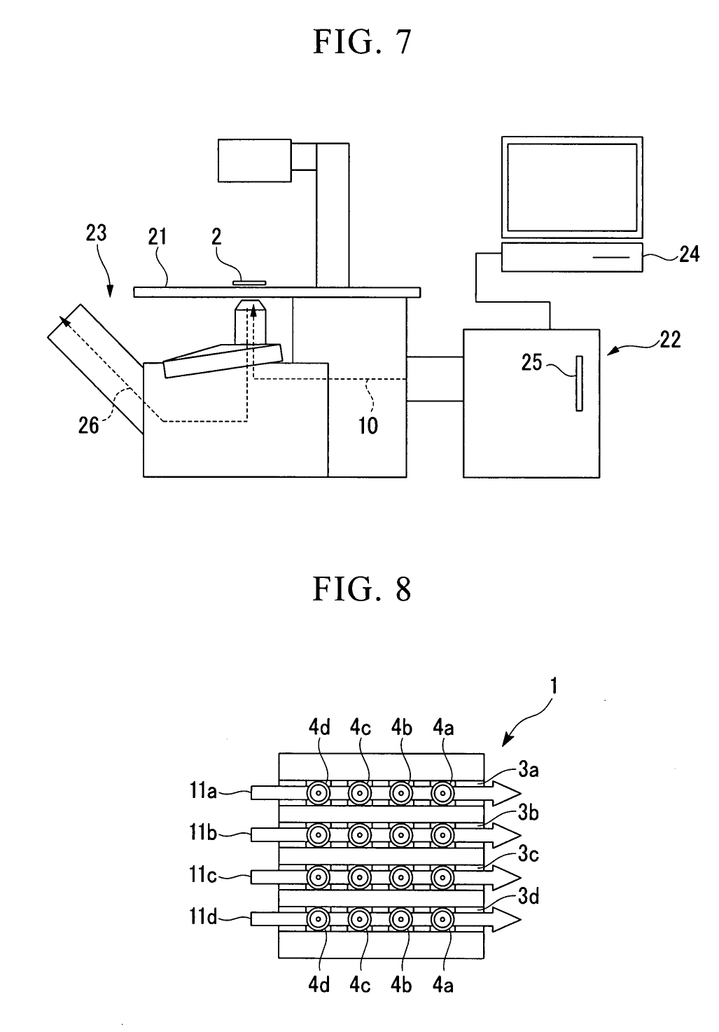

FIG. 7 is a schematic diagram showing an example of an irradiation apparatus for irradiating light to the substrate 2.

The irradiation apparatus shown in FIG. 7 has a holding platform 21 (holding unit), a irradiation unit 22 for irradiating light 10 at a predetermined region of the substrate 2, an inverted microscope 23 (observation unit) capable of observing the substrate 2, and a control unit 24 such as a personal computer.

The irradiation unit 22 is provided with a light source (not shown) and a digital micromirror device (DMD) 25 (reflection device). The DMD 25 is divided into a plurality of micromirrors. Each of the micromirrors is arranged so that the angle thereof can be independently set by the signal from the control unit 24, and reflects light from the light source to irradiate the substrate 2 with the light 10 having a pattern corresponding to the signal. Thus, by the constitution as described above, the DMD 25 can irradiate the light 10 at a predetermined region of the substrate 2. For example, the light 10 can be irradiated at one region of the surface of the substrate 2, or the entire region of the substrate surface can be irradiated with the light 10.

As the light source, a typical ultraviolet lamp can be used.

The inverted microscope 23 enables observation of cells on the substrate 2 by observation light 26.

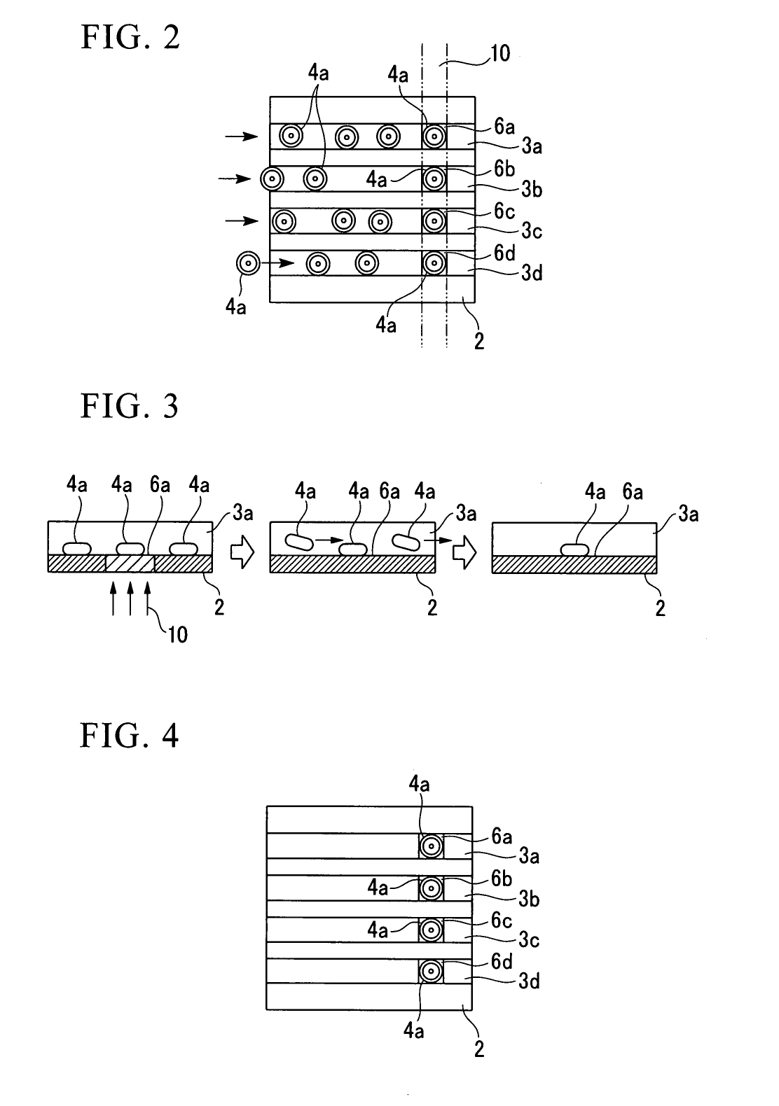

As shown in FIG. 2, a culture solution containing the first cells 4a is introduced into the first through fourth flow channels 3a to 3d of the substrate 2.

As the culture solution, a cell culturing media which is capable of maintaining a good physiological state of the cells 4a to 4d can be used. As the media, a typical base media to which a serum has been added can be exemplified. Examples of a base media include D'MEM, HamF12, HamF10 and RPMI1640. These media can be used individually, or two or more may be mixed together.

As the serum, one or more of FBS (Fetal Bovine Serum), FCS (Fetal Calf Serum), NCS (Newborn Calf serum), CS (Calf Serum), and HS (Horse Serum) can be used.

Alternatively, as the media, a serum-free media or a protein-free media can be used.

Examples of cells usable in the present invention include animal cells (e.g., human cells), plant cells and microbe cells.

Subsequently, as shown in FIGS. 2 and 3, a portion of the flow channels 3a to 3d is irradiated with the light 10 using the above-mentioned irradiation apparatus. In the shown example, the light 10 is linearly irradiated along the direction perpendicular to the flow channels 3a to 3d. Further, in the shown example, the light 10 is irradiated from the lower side of the substrate 2 (i.e., the side opposite to the side where cells 4a to 4d are present), and the light 10 is transmitted through the bottoms of the flow channels 3a to 3d to reach the cells 4a.

When the wavelength of the light 10 irradiated at the flow channels 3a to 3d is too short, the light 10 harmfully affects the physiological state of the cells 4a to 4d. On the other hand, when the wavelength of the light 10 is too long, the adhesion of the cells 4a to 4d becomes unsatisfactory.

For this reason, the light 10 includes light having a wavelength of 330 to 410 nm.

By using light having a wavelength within the above-mentioned range, the cells 4a to 4d can be satisfactorily adhered to the substrate 2 without damaging the cells 4a to 4d. Further, by using such light, the extracellular matrix and the membrane protein of the cells 4a to 4d are not harmfully affected by the light irradiation.

The light 10 may include light having a wavelength outside the above-mentioned range. However, wavelengths below the lower limit of the above-mentioned range (i.e., wavelengths below 330 nm) have a possibility of harmfully affecting the physiological state of the cells. Therefore, it is desirable that the intensity of the light be low.

When the irradiation energy of the light 10 is too small, the adhesion of the cells 4a to 4d becomes unsatisfactory. On the other hand, when the irradiation energy is too large, the physiological state of the cells 4a to 4d is harmfully affected. Therefore, the irradiation energy of the light 10 having a wavelength within the above-mentioned range is in the range of 1 to 100 J/cm² (preferably 1 to 70 J/cm²).

By setting the irradiation energy within the above-mentioned range, the cells 4a to 4d can be satisfactorily adhered to the substrate 2 without damaging the cells 4a to 4d.

When the intensity of the light 10 is too small, the adhesion of the cells 4a to 4d becomes unsatisfactory. On the other hand, when the intensity is too large, the physiological state of the cells 4a to 4d is harmfully affected. Therefore, the intensity of the light 10 is preferably within the range of 0.01 to 1 W/cm².

At the portions of the flow channels 3a to 3d where the light 10 is irradiated (hereafter, these portions are referred to as “first irradiation portions 6a to 6d”), the cells 4a to 4d respectively contacting the inner surfaces (bottoms) of the flow channels 3a to 3d are strongly adhered to the inner surfaces of the flow channels 3a to 3d and immobilized.

The cells 4a to 4d are strongly adhered to the flow channels 3a to 3d even when the temperature of the substrate 2 is hardly changed by the irradiation of the light 10.

As shown in FIG. 4, by washing the flow channels 3a to 3d with washing water, the first cells 4a which have not been adhered are removed from the flow channels 3a to 3d, and only the first cells 4a adhered to the first irradiation portions 6a to 6d remain. As the washing water, for example, a phosphate buffer solution can be used.

The mechanism of how the cells 4a to 4d are respectively adhered to the inner surfaces of the channels 3a to 3d by irradiation of the light 10 has not been elucidated yet, but it is presumed as follows.

The cells 4a to 4d respectively contacting the flow channels 3a to 3d secrete extracellular matrix. By the irradiation of the light 10, the molecular structure of the extracellular matrix changes, so that the properties of the extracellular matrix change to strongly adhere the cells 4a to 4d respectively on the inner surfaces of the flow channels 3a to 3d.

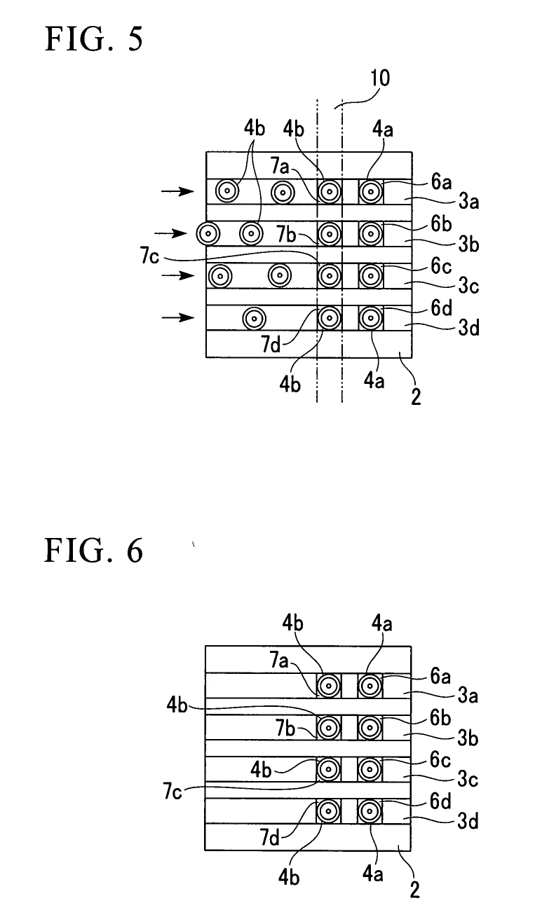

Subsequently, as shown in FIG. 5, portions of the flow channels 3a to 3d which are different from the first irradiation portions 6a to 6d (second irradiation portions 7a to 7d) are irradiated with the light 10, while introducing a culture solution containing second cells 4b into the flow channels 3a to 3d. In the shown example, the second irradiation portions 7a to 7d are more upstream of the flow of the drug agent (described below) than the first irradiation portions 6a to 6d.

In this manner, the second cells 4b are adhered and immobilized at the second irradiation portions 7a to 7d.

As shown in FIG. 6, among the second cells 4b, only those adhered to the second irradiation portions 7a to 7d remain in the flow channels 3a to 3d following washing.

Subsequently, portions of the flow channels 3a to 3d which are different from the first irradiation portions 6a to 6d and the second irradiation portions 7a to 7d (hereafter, these portions are referred to as “third irradiation portions 8a to 8d”. In the shown example, the third irradiation portions 8a to 8d are more upstream than the second irradiation portions 7a to 7d (see FIG. 1)) are irradiated with the light 10, while introducing a culture solution containing third cells 4c into the flow channels 3a to 3d, thereby adhering and immobilizing the third cells 4c at the third irradiation portions 8a to 8d.

Subsequently, portions of the flow channels 3a to 3d which are different from the first irradiation portions 6a to 6d, the second irradiation portions 7a to 7d and the third irradiation portions 8a to 8d (hereafter, these portions are referred to as “fourth irradiation portions 9a to 9d”. In the shown example, the fourth irradiation portions 9a to 9d are more upstream than the third irradiation portions 8a to 8d (see FIG. 1)) are irradiated with the light 10, while introducing a culture solution containing fourth cells 4d into the flow channels 3a to 3d, thereby adhering and immobilizing the fourth cells 4d at the fourth irradiation portions 9a to 9d.

The first through fourth cells 4a to 4d may be cells of different types.

By the procedure as described above, a cell array 1 in which cells 4a to 4d are respectively adhered to the four flow channels 3a to 3d can be obtained (see FIG. 1).

The irradiation of the light 10 does not physiologically damage the cells 4a to 4d, so that the cells 4a to 4d following the irradiation are maintained in a normal state. The viability of the cells 4a to 4d following the irradiation of the light 10 is 90% or more of the viability prior to irradiation.

Next, explanation is given of one example of a method for testing the action of a drug on cells using a cell array 1.

As shown in FIG. 8, first through fourth drug-containing liquids 11a to 11d are respectively introduced into the flow channels 3a to 3d. Each of the drug-containing liquids 11a to 11d preferably contains a drug different from those contained in the other drug-containing liquids.

Thus, each of the first through fourth drug-containing liquids 11a to 11d contacts the first through fourth cells 4a to 4d, so that assays of all combinations of the 4 types of drugs with the 4 types of cells, namely, 16 patterns of assays, can be simultaneously performed.

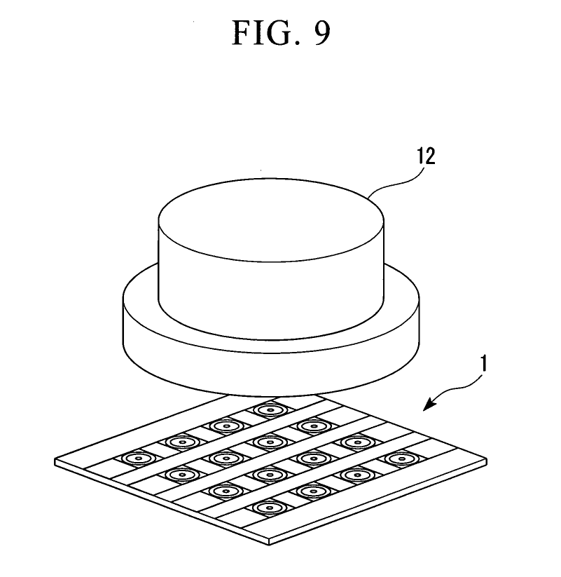

Subsequently, as shown in FIG. 9, the reactions of the drug-containing liquids 11a to 11d with the cells 4a to 4d are detected by a detection device 12.

The method for the detection is not particularly limited. For example, a method can be employed in which the drug-containing liquids 11a to 11d are labeled with a fluorescent dye or a radioactive substance, and the amount of the labeled drug taken up by the cells 4a to 4d is detected by the intensity of fluorescence.

Alternatively, the following methods can be employed: a method in which GFP (Green Fluorescent Protein) gene is introduced into the cells 4a to 4d, and the amount of GFP generated is detected on the basis of the fluorescence intensity; a method in which, using a label exhibiting fluorescence by the enzyme activity such as an esterase, the viability of the cells is detected by fluorescence intensity; and a method in which the physiological activity of the cells is detected by immunostaining physiologically active substances generated by the cells.

In the above-mentioned method for immobilizing cells, the cells 4a to 4d are adhered to the inner surfaces of the flow channels 3a to 3d by irradiating the light 10 including light having a wavelength of 330 to 410 nm, so that the cells 4a to 4d can be satisfactorily adhered and immobilized on the substrate 2 without physiologically damaging the cells.

Thus, an accurate measurement can be performed in testing the action of a drug on cells 4a to 4d.

Further, since the cells 4a to 4d can be adhered to the substrate 2 without any intermediate substance such as antibodies or an organic compound membrane, there is no need for a pretreatment step of the substrate 2. Thus, the operation can be simplified, and the cells 4a to 4d can be efficiently immobilized. Consequently, the production cost of the cell array 1 can be reduced.

When an intermediate substance such as antibodies is used, it is highly possible that the intermediate substance adversely affects the physiological state of the cells. However, in the above-mentioned method for immobilizing cells, since the cells 4a to 4d are adhered to the substrate 2 without an intermediate substance, there is no danger of the physiological state of the cells being harmfully affected.

Thus, an accurate measurement can be performed in testing the action of a drug on cells 4a to 4d.

Furthermore, since the cells 4a to 4d are directly adhered to the substrate 2, the number of steps in the operation can be decreased, so that contamination hardly occurs.

In the testing method using a cell array 1, by immobilizing a plurality of types of cells 4a to 4d in a plurality of flow channels 3a to 3d, assays can be simultaneously performed with respect to all combinations of the cells 4a to 4d with the drug-containing liquids 11a to 11d. Therefore, a multitude of assays can be efficiently performed, and the action of a plurality of types of drug-containing liquids 11a to 11d can be studied easily at low cost.

Consequently, by producing a cell array 1 using a user's cells 4a to 4d, it becomes possible to individually comply with the user's characteristics. For example, in medical applications, it becomes possible to perform medical treatment based on the characteristics (e.g. drug sensitivity) of individual patients.

Further, in the prior art, the operation of arranging cells on a microarray chip was performed by spotting in a open system, so that it was difficult to avoid contamination. However, in the method using a cell array 1, the sequence of operation can be performed in a closed system of the flow channels 3a to 3d.

Consequently, contamination can be avoided, and accurate assays can be performed under an aseptic condition.

In the above-mentioned testing method, the cells 4a to 4d which differ from each other are adhered to the flow channels 3a to 3d. However, in the present invention, it is satisfactory if 2 or more of the plurality of cells differ from each other in at least one of the plurality of flow channels.

Further, in the above-mentioned testing method, different drug-containing liquids 11a to 11d are respectively introduced into the flow channels 3a to 3d. However, in the present invention, it is satisfactory if different drug-containing liquids are introduced into at least 2 flow channels.

Next, an explanation is given of one example of the method for sorting cells according to the present invention.

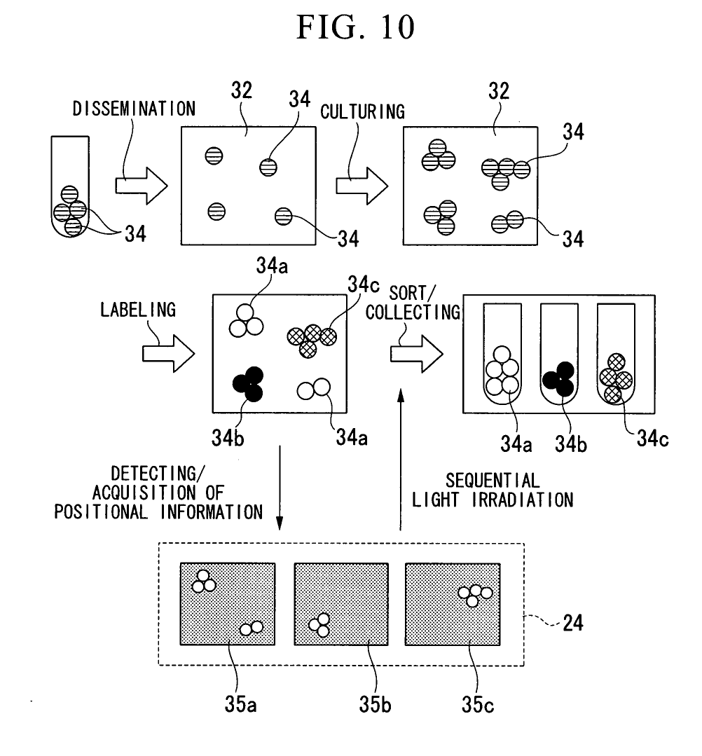

As shown in FIG. 10, a substrate 32 such as a culturing dish or a culturing cuvette is prepared. As the material for the substrate 32, those exemplified above for the substrate 2 can be used.

On the surface of the substrate 32, cells 34 including a plurality of types of cells 34a to 34c are disseminated and cultured. The cells 34a to 34c proliferate on the surface of the substrate 32.

Then, for example, polyclonal or monoclonal antibodies labeled with a fluorescent dye or the like are added and are allowed to bind to the cells 34a to 34c. By adding and binding the antibodies, it becomes possible to detect the positional information of the cells to be sorted.

Subsequently, the positional information 35a to 35c of the cells 34a to 34c are acquired by the control unit 24 of the irradiation apparatus shown in FIG. 7, and, based on the positional information 35a to 35c, light 10 is irradiated only at target cells.

The cells irradiated with the light 10 are immobilized on the substrate 32, so that cells 34a to 34c can be sorted and collected by, for example, washing off the cells which have not been irradiated with the light 10. More specifically, when cells 34a are to be collected, only the cells 34b and 34c are irradiated with the light 10 to immobilize these cells on the substrate 32, and then, it becomes possible to collect only the cells 34a by washing.

In the present invention, various fluorescent labeling methods may be used as well as the above method using a fluorescent dye. For example, a polynucleotide encoding an enzyme constituting a luminous system, such as luciferase, may be introduced into a cell. Further, for sorting cells having different morphologies, target cells may be sorted and collected by light irradiation under microscopic observation, without particular fluorescence labeling.

According to the present invention, desired cells can be selected from cells cultured on a substrate and immobilized thereon, thereby patterning the desired cells. That is, greatly differing from the conventional patterning in which a culturing substrate having supported thereon a cell-adhesive substance following a desired pattern is used, in the present invention, cells to be immobilized can be selected after culturing.

In the above-mentioned method, cells are maintained in a normal state even after light irradiation. Therefore, in the above-mentioned method, cells having specific characteristics, such as cells exhibiting high generation efficiency of physiologically active substances or cells in which stable transfer is confirmed following gene transfer, can be applied to an operation in which the cells are purified, and successively cultured to proliferate the cells.

Next, an explanation is given of modifications of the cell sorting method according to the present invention. In the explanation below, with respect to the constitutions which have already been explained above, the same reference numerals are used, and explanations thereof are omitted.

In an axenic culture, it is necessary that invasion of unwanted bacteria be avoided. However, when invasion of unwanted bacteria occurs, the unwanted bacteria can be removed as follows.

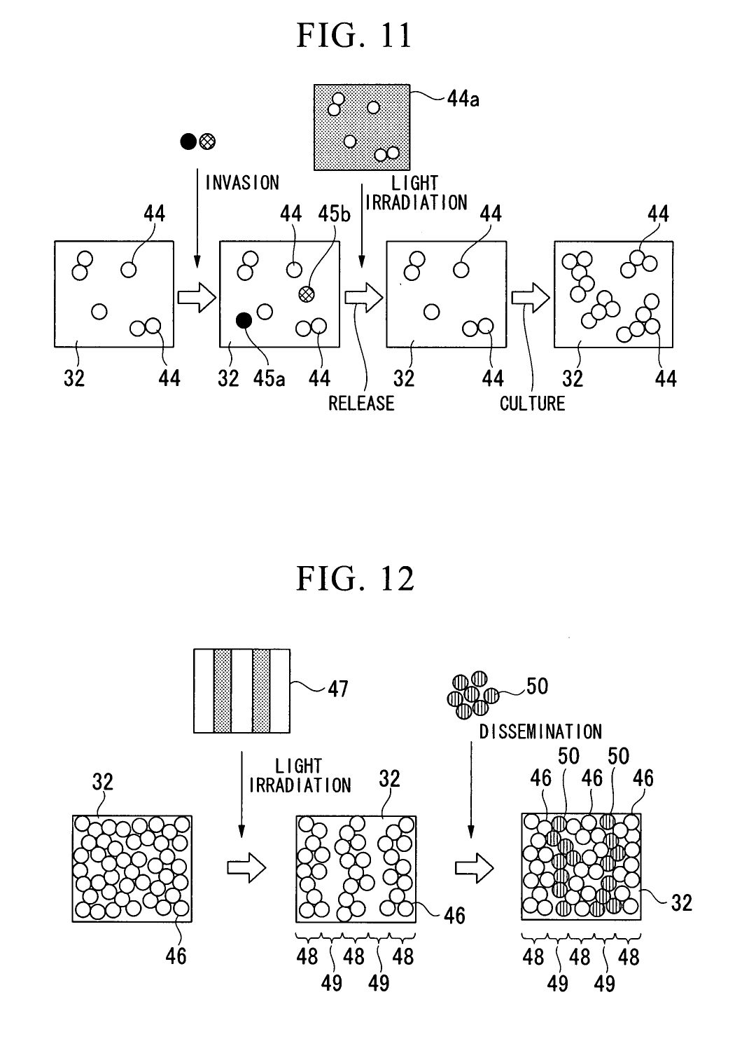

As shown in FIG. 11, specific cells 44 are cultured on the surface of a substrate 32. When the substrate 32 is invaded by other cells 45a and 45b, the positional information 44a of the specific cells 44 obtained by the aforementioned fluorescent labeling method is acquired by the control unit 24 of the irradiation apparatus shown in FIG. 7. Then, based on the positional information 44a, light 10 is irradiated onto only the specific cells 44 to immobilize the specific cells 44 on the substrate 32, and the invading cells 45a and 45b are released from the substrate 32 by washing or the like, thereby removing the invading cells 45a and 45b.

In a case where the specific cells 44 and the invading cells 45a and 45b can be distinguished by visual observation, the irradiation pattern of the light 10 can be determined by microscopic observation without labeling.

The adhesion of cells by light irradiation may weaken with time. For example, when cells adhered to a substrate by light irradiation are left to stand for a predetermined period of time following the light irradiation, the cells may become releasable again. Therefore, for the purpose of removing unwanted bacteria, desired cells may be temporary adhered to a substrate, and then sorted and collected by the above-mentioned cell sorting method.

FIG. 12 is a flow chart showing a process of patterning proliferation regions of cells.

First cells 46 are proliferated over the entire surface of a substrate 32, and light 10 is irradiated onto first regions 48 in accordance with a pattern 47, thereby immobilizing the first cells 46 located in the first regions 48 of the substrate 32. The first cells 46 located in the other regions (second regions 49) are removed by washing or the like. In the shown example, the first regions 48 are formed linearly and in parallel to each other.

By proliferating second cells 50 in the second regions 49 from which the first cells 46 have been removed, the first regions 48 in which the first cells 46 are present and the second regions 49 in which the second cells 50 are present are alternately arranged in a predetermined direction (in a crosswise direction in the figure).

Such patterning of proliferation regions of cells is effective in analyzing signal transduction of cells, or producing physiologically active substances generated under co-existence of a plurality of types of cells.

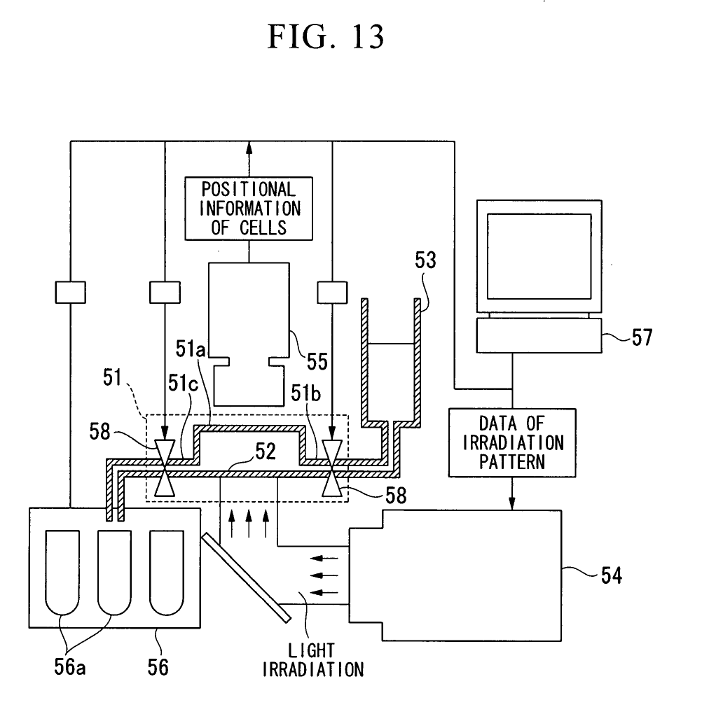

FIG. 13 is an example of an apparatus usable for sorting cells.

The cell sorting apparatus shown in the figure is constituted of: a cell culturing unit including a substrate to which the cells can be adhered; a culture-medium supplying unit for supplying culture medium to the cell culturing unit; an irradiation unit for irradiating light to the substrate; a cell-position detecting unit for detecting the position of cells on the substrate; a sorting unit for sorting cells released by light irradiation; and optionally a washing-water supplying unit.

Hereinbelow, the constitution of this cell sorting apparatus is described in detail.

The cell sorting apparatus is provided with: a cell culturing cuvette 51 (cell culturing unit) including a substrate 52 to which cells can be adhered; a culture medium reservoir 53 (culture-medium supplying unit) for supplying a culture medium to the cuvette 51; a projector 54 (irradiation unit) for irradiating light 10 to the substrate 52 of the cuvette 51; a color CCD camera 55 (cell-position detecting unit) for detecting respective positions of cells and transmitting a signal of the detected positional information to a control unit 57; a sorting/collection device 56 including a plurality of switchable collecting vessels 56a; and the control unit 57 for controlling the above-mentioned units/device.

If desired, the cell sorting apparatus may be provided with a washing-water supplying unit (not shown) for supplying washing water to the cell culturing cuvette 51.

The cell culturing cuvette 51 is a vessel having a bottom made of the substrate 52, and the substrate 52 can be externally irradiated with the light 10, so as to observe cells on the surface of the substrate 52.

The cell culturing cuvette 51 includes a main part 51a having the substrate 52, an inlet channel 51b for introducing a culture medium to the main part 51a, and an outlet channel 51c for discharging the culture medium. The channels 51b and 51c are provided with switching devices 58 such as valves for opening and closing the channels. As the material for the substrate 52, any of those exemplified above for the substrate 2 may be used.

The projector 54 is an irradiation unit for irradiating light having a pattern corresponding to the signal from the control unit 57, and is capable of irradiating a desired region of the surface of the substrate 52.

The projector 54 includes a light source (not shown) and an optical conversion part (not shown) for converting the pattern of the irradiation region of the light irradiated from the light source into the pattern corresponding to the signal from the control unit 57. As the optical conversion part, a digital micromirror device (DMD) or a transparent liquid crystal panel can be used.

It is desirable that the control unit 57 be capable of acquiring the positional information of the cells. Further, it is desirable that the control unit 57 be capable of controlling the setting of the light irradiation from the projector 54, as well as suppliance and stoppage of the culture medium or washing water to the cell culturing cuvette 51, based on the positional information acquired.

The color CCD camera 55 preferably has sufficient resolution, optical magnification and sensitivity for distinguishing individual cells or cell colonies. Further, when a plurality of antibody-supporting fluorescent dyes is used to distinguish the types of cells, it is desirable that the color CCD camera be capable of distinguishing color.

In the description above, explanation has been made of a process in which cells are disemminated and cultured in a cell-culturing cuvette. However, the present invention can be applied to a process in which cells are simply introduced into a cuvette, followed by sorting and collecting of the cells. Such process is effective in sorting and collecting cells from a tissue containing a plurality of types of cells.

Next, explanation is made of one example of operation of the above-mentioned cell sorting apparatus.

By opening and closing the switching device 58, culture is supplied from the culture reservoir 53 to the cell-culturing cuvette 51, and cells are disseminated and cultured in the cuvette.

Among the cells, the target cells may be labeled with fluorescence in advance, or labeled with fluorescent antibodies following cell culturing. The respective positions of the cells on the substrate 52 of the cell-culturing cuvette 51 are detected by the color CCD camera 55, and the positional information obtained is entered into the control unit 57.

Thus, the target cells to be sorted are distinguished by the fluorescence labeling, and light 10 is irradiated by the projector 54 at cells other than the target cells. The position to be irradiated can be automatically adjusted by the control unit 57, or manually adjusted while observing the cells.

The cells irradiated by the light 10 adhere to the substrate 52, whereas the cells which have not been irradiated are releasable from the substrate 52. Therefore, cells can be selectively removed from the substrate 52 by supplying a culture medium or washing water to the cell-culturing cuvette 51, and then collected in a collecting vessel 56a of the sorting/collection device 56.

The sorting/collection device 56 is provided with a plurality of switchable collecting vessels 56a, so that the sorted cells of different types can be respectively collected in the collecting vessels 56a.

### EXAMPLES

### Example 1

A plate-like substrate (96 wells) made of a plasma-treated polystyrene (TCPS) was prepared, and animal cells were disseminated in an average number of 100 cells per well, and cultured for 23 hours. Then, using the irradiation apparatus shown in FIG. 7, light was irradiated from the bottom side of the wells under various conditions of wavelength and energy. As the animal cells, CHO-K1 cells were used.

Subsequently, the surface of the substrate washed with a phosphate buffer solution containing 1 mM of EDTA, and the amount of cells remaining was visually observed, so as to evaluate the cell adhesion induced by the light irradiation. The evaluations of cell adhesion are indicated in Tables 1 and 2 with the following criteria: (A) 80% or more cells remaining following a predetermined washing process sufficient for removing unirradiated cells; (B) 50% or more to less than 80% of the cells remaining; (C) 20% or more to less than 50% of the cells remaining; (D) less than 20% of the cells remaining.

The proliferation ability of the cells was evaluated as follows. After 3 days from the light irradiation, the cells were subjected to a freezing treatment. Then, CyQUAUT exhibiting fluorescence having an intensity proportional to the number of cells was added, and the fluorescence intensity was measured by a plate reader. By comparing the fluorescence intensity with the fluorescence intensity of an unirradiated sample, the proliferation ability of the cells was evaluated. The evaluations of the proliferation ability of cells are indicated in Tables 1 and 2 with the following criteria: (A) intensity of 90% or more of the unirradiated sample; (B) intensity of 50% or more to less than 90% of the unirradiated sample; (C) intensity of less than 50% of the unirradiated sample.

TABLE 1

| | | Irradiation | Light | Irradiation | | Proliferation |
|---|---|---|---|---|---|---|
| | Wavelength | energy | intensity | time | Cell | ability of |
| | (nm) | (J/cm²) | (W/cm²) | (seconds) | adhesion | immobilized cells |
| Test | 313 | 2 | 0.067 | 30 | — | C |
| Example 1 | | | | | | |
| Test | 334 | 1.8 | 0.06 | 30 | B | B |
| Example 2 | | | | | | |
| Test | 365 | 15 | 0.05 | 300 | A | A |
| Example 3 | | | | | | |
| Test | 405 | 70 | 0.58 | 300 | C | A |
| Example 4 | | | | | | |
| Test | 405 | 174 | 0.58 | 300 | B | B |
| Example 5 | | | | | | |
| Test | 436 | 30 | 0.1 | 300 | D | — |
| Example 6 | | | | | | |

TABLE 2

| | | Irradiation | Light | Irradiation | | Proliferation |
|---|---|---|---|---|---|---|
| | Wavelength | energy | intensity | time | Cell | ability of |
| | (nm) | (J/cm²) | (W/cm²) | (seconds) | adhesion | immobilized cells |
| Test | 365 | 0.6 | 0.01 | 60 | D | — |
| Example 7 | | | | | | |
| Test | 365 | 1.2 | 0.01 | 120 | B | A |
| Example 8 | | | | | | |
| Test | 365 | 6 | 0.05 | 120 | A | A |
| Example 9 | | | | | | |
| Test | 365 | 28 | 0.23 | 120 | A | A |
| Example 10 | | | | | | |
| Test | 365 | 69 | 0.23 | 300 | A | A |
| Example 11 | | | | | | |
| Test | 365 | 120 | 0.5 | 240 | A | C |
| Example 12 | | | | | | |

As shown in Table 1, in Test Example 1 in which light having a wavelength of 313 nm was used, a result was obtained indicating that almost all of the cells were killed.

In Test Example 2 in which light having a wavelength of 334 nm was used, adhesion of cells to the substrate surface was observed. Although no killing of cells was observed, a slight influence on the proliferation ability of the cells was observed.

In Test Example 3 in which light having a wavelength of 365 nm was used, cell adhesion was enhanced without adverse influence on the proliferation ability of the cells.

From the results of Test Examples 1 to 3, it is presumed that, when light having a wavelength shorter than that of the light used in Test Example 2, cells are markedly damaged by irradiation with light having sufficient intensity for cell adhesion.

In Test Examples 4 and 5, light having a wavelength of 405 nm was used. In Test Example 4 in which the irradiation energy was 70 J/cm², no adverse influence on the proliferation ability of cells was observed, which indicates that the damage to the cells was small. However, in Test Example 4, the cell adhesion was slightly low. On the other hand, in Test Example 5 in which the irradiation energy was larger than that in Test Example 4, although the cell adhesion was enhanced, a slight influence on the proliferation of the cells was observed.

From the above, it is presumed that, when light having a wavelength longer than that of the light used in Test Examples 4 to 5 is used, a satisfactory cell adhesion cannot be achieved by irradiation with light having intensity sufficiently low that marked influence on the proliferation ability of cells is not observed.

In Test Example 6 in which light having a wavelength of 436 nm was used, the cell adhesion was unsatisfactory.

From the above, it is proved that irradiation of light having a wavelength of 330 to 410 nm is appropriate for achieving satisfactory cell adhesion without causing adverse influence on the proliferation ability of the cells.

As shown in Table 2, in Test Example 7 in which the irradiation energy was 0.6 J/cm², the cell adhesion was unsatisfactory, whereas in Test Example 12 in which the irradiation energy was 120 J/cm², the proliferation ability of the cells was unsatisfactory.

The influence of irradiation energy of light on the proliferation ability of the cells was studied as follows.

CHO-K1 cells were cultured on the surface of a substrate. Then, the substrate surface was irradiated with light having a predetermined pattern, followed by washing with a phosphate buffer solution containing 1 mM of EDTA. The wavelength of the light was 365 nm.

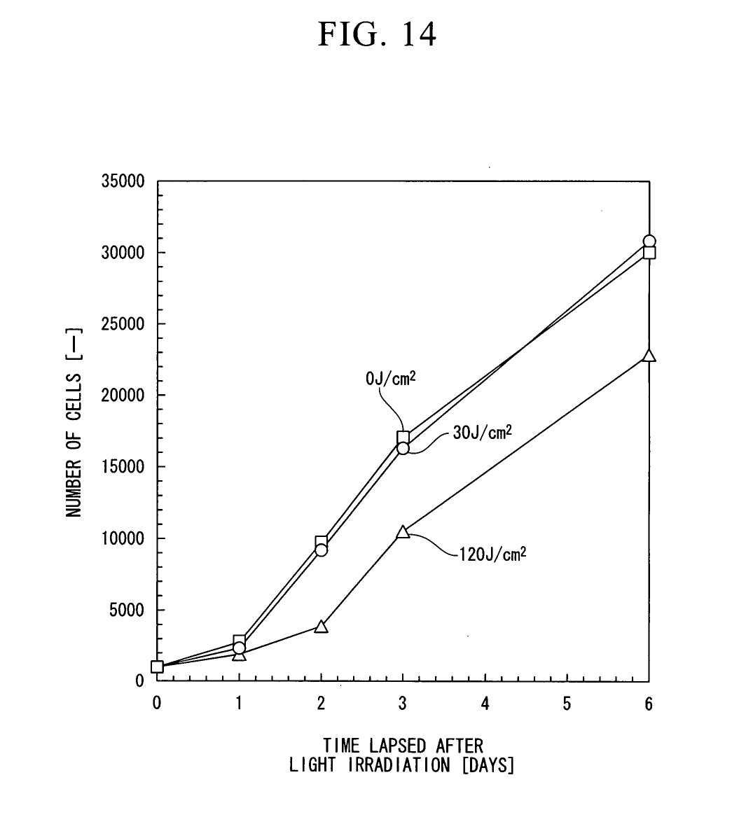

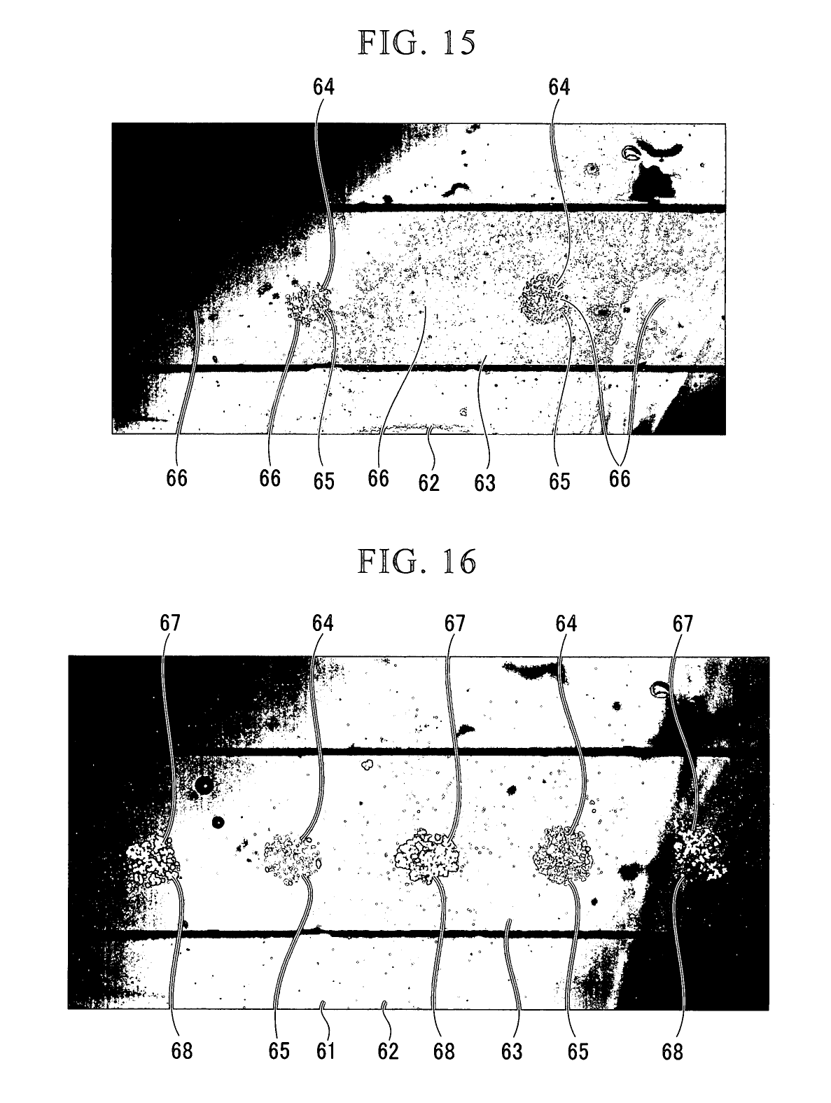

FIG. 14 is a graph showing the change with lapse of time in the number of cells following the light irradiation. The vertical axis indicates the number of cells, and the horizontal axis indicates the time lapsed following the light irradiation. The irradiation energies of light used were 30 J/cm² and 120 J/cm². For comparison, the result of a test in which light irradiation was not performed (0 J/cm²) is also shown.

In the case where the irradiation energy was 30 J/cm², the proliferation ability of the cells was the same as that in the case where light irradiation was not performed. On the other hand, in the case where the irradiation energy was 120 J/cm², the proliferation ability of the cells became poor.

From FIG. 14 and Table 2, it is proved that, by using light having an irradiation energy of 1 to 100 J/cm² (preferably 1 to 70 J/cm²), the cell adhesion can be enhanced without adversely affecting the proliferation ability of the cells.

### Example 2

Cell array 61 was manufactured as follows (see FIGS. 15 and 16). A substrate 62 made of a plasma-treated polystyrene (TCPS) was prepared, which was covered with a silicone resin inhibiting cell adhesion except for five circular regions 66 having a diameter of 200 μm. A channel 63 was formed so as to have a rectangular cross-section with a width of 600 μm and a depth of 200 μm.

A culture solution containing MDCK cells dyed with CMTPX exhibiting a red fluorescence was introduced into the channel 63, and culturing was performed for 5 hours and 30 minutes.

Subsequently, using the irradiation apparatus shown in FIG. 7, light was locally irradiated onto the channel 63, so as to immobilize MDCK cells 65 as first cells in two of the five circular regions 66 (first irradiation portions 64). The remainder of the cells, namely, cells which had not been immobilized, were removed by washing.

FIG. 15 is a fluorescent microphotograph of the channel 63 in which the first cells (MDCK cells 65) have been immobilized on the first irradiation portions 64.

After 2 hours of culturing, a culture solution containing CHO cells 68 dyed with CMFDA exhibiting a green fluorescence was introduced into the channel 63, and culturing was performed for 5 hours and 30 minutes.

Subsequently, using the irradiation apparatus shown in FIG. 7 again, light was locally irradiated onto the channel 63, so as to immobilize CHO cells 68 as second cells in the remaining three of the five circular regions 66 (second irradiation portions 67). The remainder of the cells were removed by washing.

FIG. 16 is a fluorescent microphotograph of the channel 63 in which the second cells (CHO cells 68) have been immobilized in the second irradiation portions 67. It can be seen that MDCK cells 65 and CHO cells 68 had been immobilized at a different position within the same channel 63.

For immobilizing MDCK cells 65 and CHO cells 68, light having a wavelength of 365 nm and an intensity of 0.026 W/cm² was used, and the irradiation time was 150 seconds (irradiation energy: 3.9 J/cm²).

### Example 3

A substrate 72 made of a plasma-treated polystyrene (TCPS) was prepared, and MDCK cells 73 were uniformly disseminated on the surface of the substrate 72 and cultured for 4 hours. Then, using the irradiation apparatus shown in FIG. 7, light was irradiated onto the substrate at a region 74 forming the characters “AIST” and a rectangular region 75. The wavelength of the light was 365 nm, the intensity was 0.08 W/cm², and the irradiation time was 10 minutes (irradiation energy: 48 J/cm²).

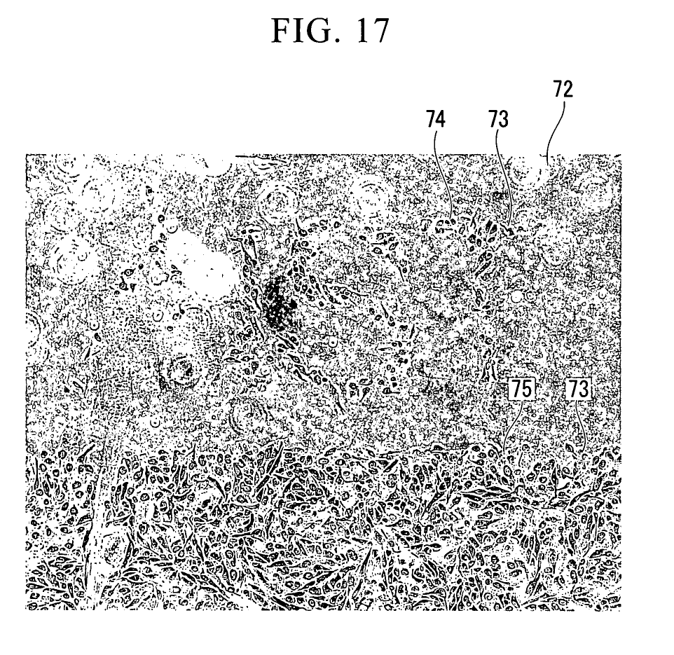

FIG. 17 is a photograph of the surface of the substrate 72 following washing with a phosphate buffer solution containing 1 mM of EDTA. It can be seen that cells 73 had been immobilized only in the regions 74 and 75 where the light was irradiated.

### Example 4

CHO-K1 cells were uniformly disseminated on a polystyrene substrate coated with fibronectin (No. 354457, manufactured by BD Bioscience), and culturing was performed for 24 hours. Then, using the irradiation apparatus shown in FIG. 7, the substrate surface was irradiated with light having a predetermined pattern. The wavelength of the light was 365 nm, and the irradiation energy was 18 J/cm².

Subsequently, a phosphate buffer solution containing 1 mM of EDTA was effected to the substrate for 10 minutes. Then, the substrate surface washed in the same manner as in Example 3.

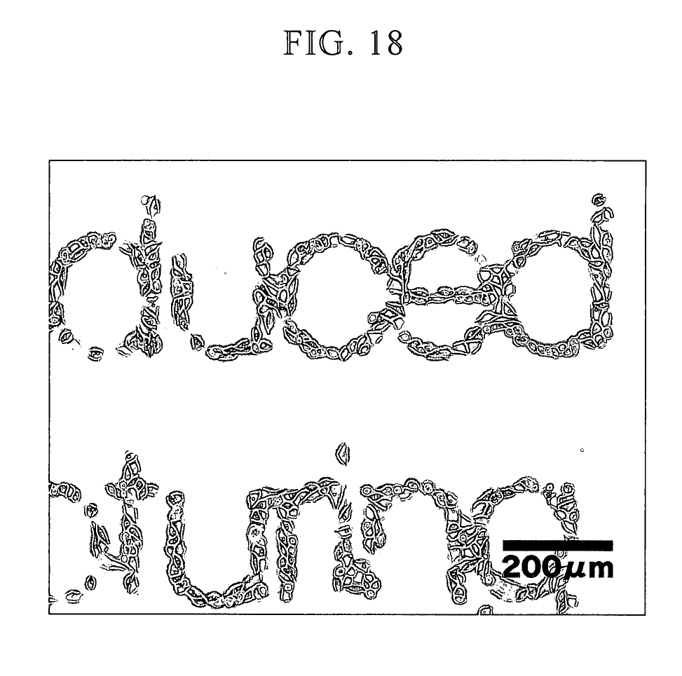

FIG. 18 is a photograph of the substrate surface following washing. It can be seen that the cells had been immobilized in the irradiated region, whereas the cells had been completely removed in almost all of the unirradiated regions. This indicates that a pattern with a high contrast was obtained.

### Example 5

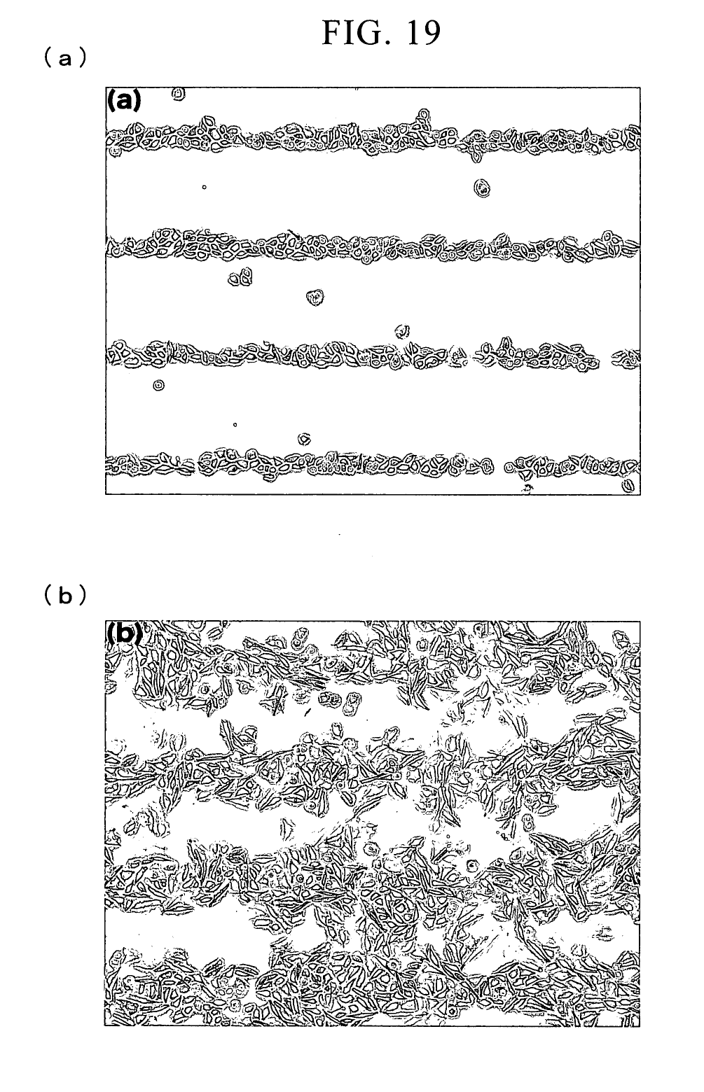

A linear pattern of CHO-K1 cells was formed on the surface of a substrate in substantially the same manner as in Example 4. FIG. 19(a) is a photograph of the substrate surface. Further, FIG. 19(b) is a photograph of the substrate surface following culturing of cells for 24 hours.

From FIGS. 19(a) and 19(b), it can be seen that the cells following the irradiation of light still maintained satisfactory viability, and that cells had proliferated to the outside of the irradiated region.

### Example 6

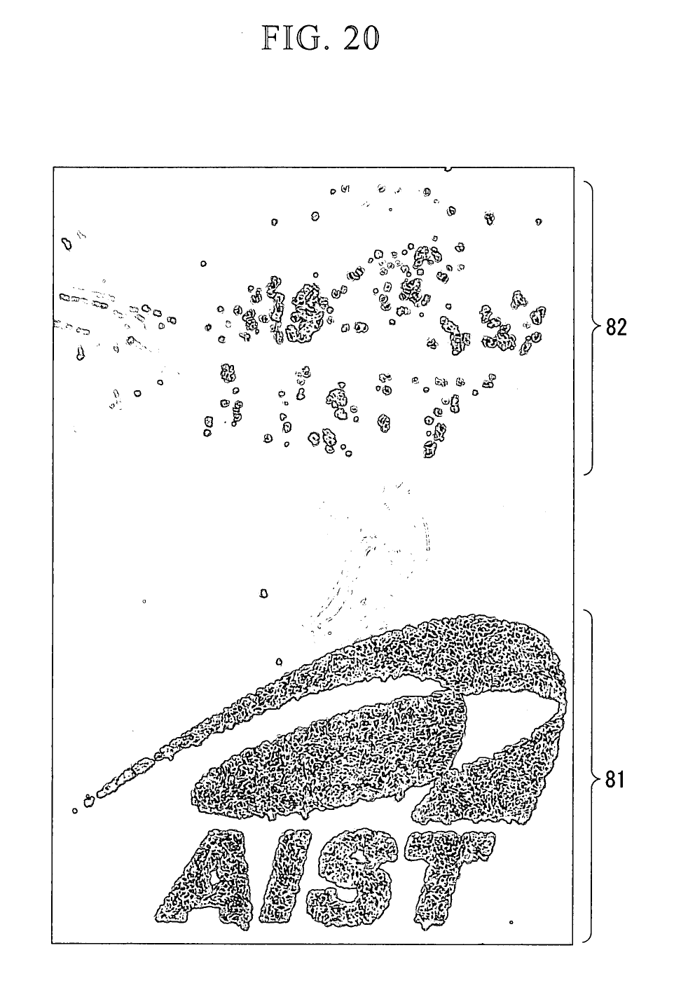

A predetermined pattern was formed in the same manner as in Example 4, as follows. CHO-K1 cells were cultured on the surface of a substrate, and light having a predetermined pattern was irradiated thereat. Immediately after the light irradiation, the substrate surface washed with a phosphate buffer solution (PBS) containing 1 mM of EDTA (flow rate of PBS during washing: 2 m/s), thereby forming a predetermined pattern (referred to as numeral 81 in FIG. 20).

Subsequently, the cells forming the above-mentioned pattern were further cultured for 8 hours, followed by washing of the substrate surface under the same conditions as mentioned above (flow rate of PBS during washing: 2 m/s). As a result, almost all of the cells were removed from the substrate surface. The numeral 82 in FIG. 20 refers to the substrate surface following washing.

This result indicates that the strength of cell adhesion by light irradiation had weakened with time.

From the above, it is proved that the adhesion and releasing of cells can be easily controlled.

### Example 7

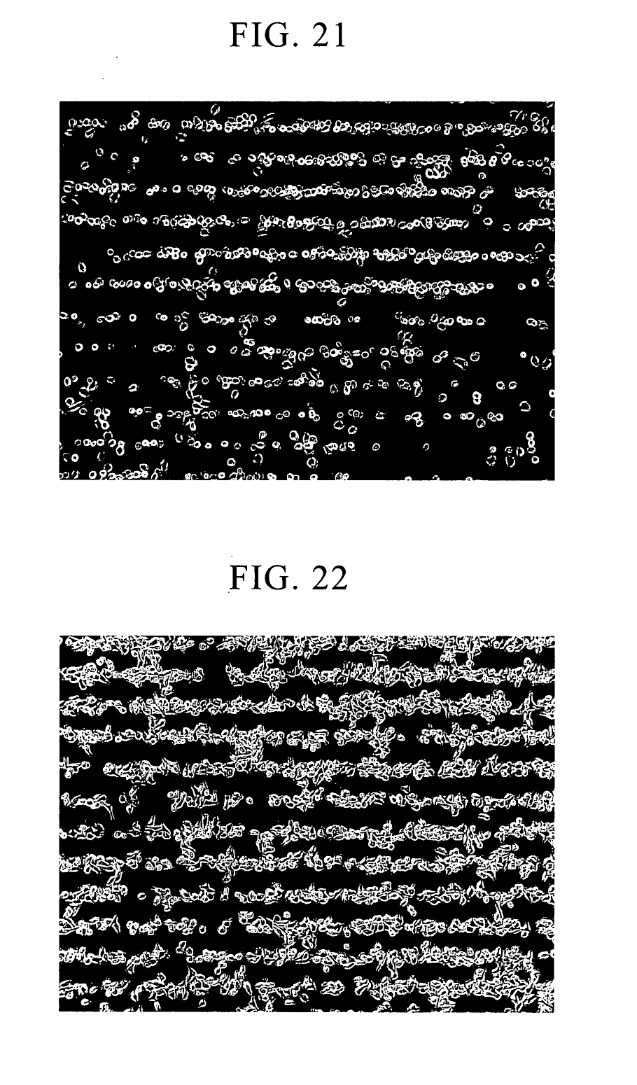

A linear pattern was formed in substantially the same manner as in Example 5, except that HeLa cells were used. FIG. 21 is a photograph of the substrate surface. The wavelength of the light was 365 nm, and the irradiation energy was 3.5 J/cm².

### Example 8

A linear pattern was formed in substantially the same manner as in Example 5, except that HepG2 cells were used. FIG. 22 is a photograph of the substrate surface. The wavelength of the light was 365 nm, and the irradiation energy was 3.0 J/cm².

### Example 9

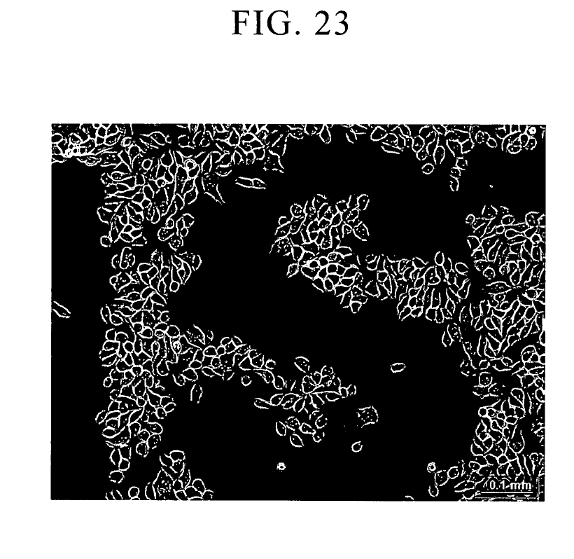

A pattern was formed in substantially the same manner as in Example 5, except that MDCK cells were used. The pattern was formed in a manner such that the portion from which the cells had been removed exhibited the character “S”. FIG. 23 is a photograph of the substrate surface. The wavelength of the light was 365 nm, and the irradiation energy was 24 J/cm².

From the results of Examples 7 to 9, it is proved that the present invention is applicable to a plurality of types of cells.

### Example 10

A pattern was formed using 2 types of cells, as follows.

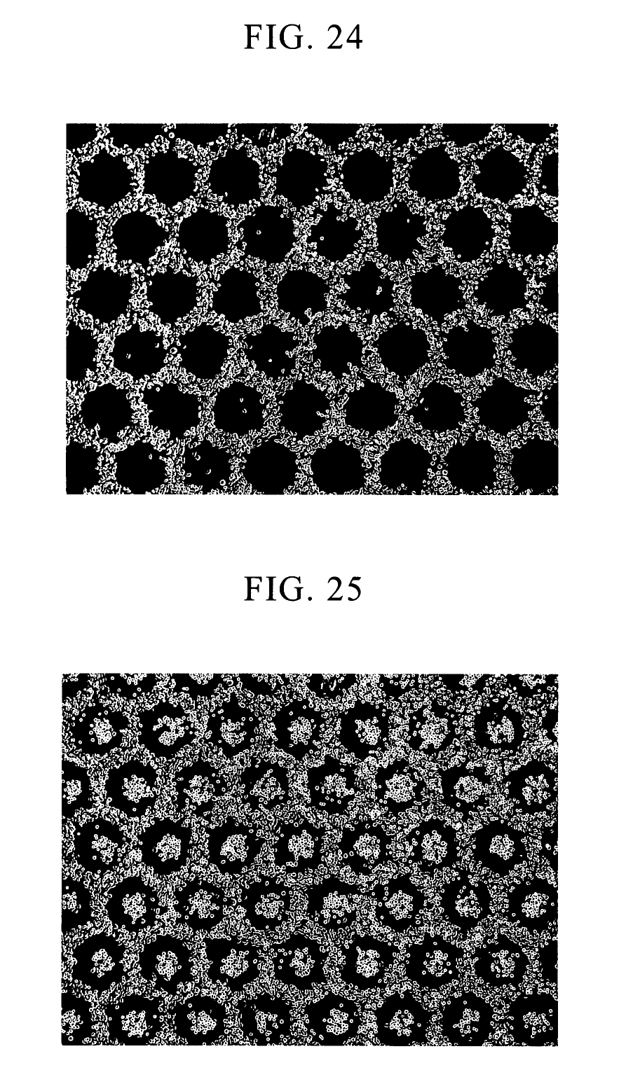

A honeycomb pattern was formed on a culturing substrate using CHO-K1 cells, in the same manner as in Example 4. FIG. 24 is a photograph of the substrate surface.

Subsequently, in the same manner, HeLa cells were respectively adhered in the form of dots in the centers of hexagons forming the above-mentioned honeycomb pattern. FIG. 25 is a photograph of the substrate surface.

Thus, cells could be additionally adhered with a predetermined pattern to a substrate which already had cells adhered.

In the method for immobilizing cells according to the present invention, cells are adhered to the surface of a substrate by irradiating light including light having a wavelength of 330 to 410 nm. Therefore, cells can be satisfactorily adhered and immobilized on the substrate without damaging the cells.

Therefore, an accurate measurement can be performed in testing the action of a drug on the cells.

Further, since the cells can be adhered to the substrate without any intermediate substance such as antibodies, there is no need for a pretreatment step of the substrate. Thus, the operation can be simplified, and the cells can be efficiently immobilized. Consequently, the production cost of the cell-immobilized substrate can be reduced.

While preferred embodiments of the invention have been described and illustrated above, it should be understood that these are exemplary of the invention and are not to be considered as limiting. Additions, omissions, substitutions, and other modifications can be made without departing from the spirit or scope of the present invention. Accordingly, the invention is not to be considered as being limited by the foregoing description, and is only limited by the scope of the appended claims.

1. A method for immobilizing cells by adhering the cells to a surface of a substrate, comprising:
irradiating cells with light while contacting said cells to a surface of a substrate, thereby adhering said cells to said substrate,
said light comprising light having a wavelength of 330 to 410 nm.

2. The method according to claim 1, wherein said light has an irradiation energy of 1 to 100 J/cm2.

3. The method according to claim 1, wherein said cells are irradiated with said light in the presence of a serum.

4. The method according to claim 1, wherein at least the surface of said substrate comprises a non-photoresponsive material.

5. The method according to claim 4, wherein at least the surface of said substrate comprises polystyrene.

6. A cell-immobilized substrate in which cells have been immobilized by the method of claim 1.

7. An apparatus for immobilizing cells by adhering the cells to a surface of a substrate,
said apparatus being provided with an irradiation unit for irradiating a desired region of said substrate,
said irradiation unit irradiating light to cells which are in contact with the surface of said substrate, thereby adhering said cells to said substrate,
said light comprising light having a wavelength of 330 to 410 nm.

8. The apparatus according to claim 7, wherein said irradiation unit comprises a light source and a reflection device,
said reflection device reflecting light generated from said light source to irradiate a desired region of said substrate.

9. A method for testing action of drug on cells using the cell-immobilized substrate of claim 6, comprising:
contacting a drug with said cells; and
detecting action of said drug on said cells.

10. A method for sorting some cells from a plurality of types of cells, comprising:
leading a plurality of types of cells to a surface of a substrate;
selectively irradiating target cells with light comprising light having a wavelength of 330 to 410 nm while contacting said target cells to the surface of said substrate, thereby adhering said target cells to said substrate; and
removing cells other than said target cells from the surface of said substrate.
